(19) United States
(12) Patent Application Publication
(10) Pub. No.: US 2026/0021291 A1
(43) Pub. Date: Jan. 22, 2026

(54) Percutaneous Wire Lead Anchor

(71) Applicant: Amitabh Goel, Lakewood Ranch, FL (US)

(72) Inventor: Amitabh Goel, Lakewood Ranch, FL (US)

(21) Appl. No.: 19/339,737

(22) Filed: Sep. 25, 2025

(51) Int. Cl. A61N 1/05; A61N 1/372

(52) U.S. Cl. CPC A61N 1/0558; A61N 1/0502; A61N 1/372

(57) ABSTRACT

An adaptor for anchoring wire lead components of electrical nerve stimulation assemblies, the adaptor incorporating a left jaw; the adaptor further incorporating a right jaw; the adaptor further incorporating a left arm having a proximal end, a distal end, and a right side; the adaptor further incorporating a right arm; the adaptor further incorporating an upper left channel opening rightwardly; the adaptor further incorporating an upper right channel opening leftwardly; the adaptor further incorporating a lower left channel opening rightwardly; the adaptor further incorporating a lower right channel opening leftwardly; and the adaptor further incorporating a hinge which is fixedly attached to or formed wholly with the proximal ends of the left arm, the right arm, the left jaw, and the right jaw.

### FIELD OF THE INVENTION

The instant invention relates to assemblies, for electric pulse suppression of pain of the type which percutaneously extend an electrical wire lead and electrode head combination along a patient's spinal canal for the placement of the electrode head at a targeted position overlying the patient's spinal cord. More particularly, the instant invention relates to fasteners and adaptors which anchor such wire lead for resisting subsequent slippage of the wire lead and electrode head.

### BACKGROUND OF THE INVENTION

The human spinal cord includes separate and discrete nerve pathways which transmit signals, including pain signals, to the brain from the body, and from the body to the brain. To achieve pain suppression via targeted electrical pulses directed to the spinal cord, a small electrode bearing head, configured either as a narrow cylinder or a flat wafer, may be extended to and placed at a selected position over the surface of the dura mater which covers the patient's spinal cord. Upon effecting a precisely targeted placement of such electrode head, electrical pulses emanating from the electrodes may affect the transmission of the signals which are responsible for neural transmission of the subject's pain. Such precise extension and placement of the electrode head may effectively interfere with the spinal cord's transmission of the pain, and without energizing or interfering with the function of adjacent neural signal transmission tracks. Accordingly, such precise electrode head placement and electric pulse emitting operation may advantageously counteract the pain without impairing other functions.

To introduce and place such an electrode head at a precisely determined location over the patient's spinal cord dura mater, the physician may insert a hollow bored Tuohy needle into the back of the patient, causing the cutting point of such needle to pierce and traverse epidermis, dermis, and hypodermis layers of the patient's skin. The physician may cause the needle to subsequently traverse the thoracolumbar facia tissues which cover the patient's back muscles, and to further traverse underlying muscle layers which directly overlie the patient's spine. Further insertion of the Tuohy needle may pierce interspinous ligaments spanning between spinal processes of the patient's vertebra, allowing the pointed end of the needle to enter the patient's epidural space, adjacent to the spinal cord. Upon such Tuohy needle insertion, a hollow bored pathway from the patient's exterior to and into the epidural space which overlies the patient's spinal cord dura mater is established. Such needle insertion advantageously provides access to the patient's spinal canal at a selected wire lead insertion site along the spinal cord without piercing the spinal cord's protective dura mater covering.

Following use of such Tuohy needle to establish a spinal canal access pathway, the physician may thread the electrically conductive wire lead having an electrode head at its distal or longitudinal end through the needle's hollow bore to enter the epidural space. By precise threading, advancing, and positioning of such wire lead, the electrode head may be properly positioned for emissions of pain suppressing electrical pulses at and through a targeted track within the patient's spinal cord. Testing electrical pulses combined with reports of sensations made by the patient during the electrode head insertion procedure may assist the physician in establishing the desired precisely located position of the electrode head.

Following achievement of such desired precise placement of the electrode head, the Tuohy needle is commonly rearwardly withdrawn over the electrically conductive wire lead. Thereafter, the wire lead is desirably anchored upon back tissues which dorsally overlie the wire lead's point of entry into the spinal canal. Anchoring of the wire lead is typically needed because slight movements or slippage of the lead wire and electrode head may nullify the electrode head's ability to suppress the targeted pain sensations. Anchoring of the lead wire is commonly established at a zone or area underlying the patient's epidermis and dermis and immediately overlying the thoracolumbar facia covering of the patient's back musculature, through which the lead is traversing.

Commonly known wire lead anchoring adaptors which are utilized for securing electrical stimulation lead wires against slippage are commonly installed with difficulty, and such adaptors commonly insecurely grasp and hold the wire lead. Such adaptors also are subject to slippage following surgical implantation.

The instant inventive adaptor for percutaneous anchoring of a wire lead component of an electrical nerve stimulation assembly solves or ameliorates such problems by providing an adaptor which may initially laterally receive the wire lead and may subsequently securely grasp the lead wire. The inventive adaptor also includes specialized components and features for firmly anchoring the adaptor against a patient's thoracolumbar fascia.

### BRIEF SUMMARY OF THE INVENTION

The instant invention functions as an anchoring adaptor for securing against slippage of a wire lead component of an electrical nerve stimulation assembly. During a surgical implantation of such assembly within a patient's back, the patient normally lies in a prone position. The instant inventive adaptor has a longitudinal or forward end which is considered to be directionally consistent with the patient's head, cranial, or superior end. The adaptor's oppositely longitudinal or rearward end oppositely faces or points toward the patient's feet or inferior bodily end. References herein to the adaptor's downward or lower end coincide with a prone patient's ventral or anterior side, and upper ends of the adaptor's components coincide with the direction of the dorsal side of the patient's back. With such longitudinal, oppositely longitudinal, upward, downward, forward, and rearward spatial orienting directions established, the adaptor's left and right sides or ends directionally coincide with the patient's left and right arms.

A first structural component of the instant inventive adaptor comprises a hinge which establishes a longitudinally extending pivot axis. In a preferred embodiment, the adaptor's hinge component comprises a plastic living hinge. Other common types of hinges such as pin-sleeve-and-eye hinges are considered to fall within the scope of the invention.

Further structural components of the instant inventive adaptor comprise left and right jaws, each such jaw having an upper proximal end and a wire lead grasping distal end. In the preferred embodiment, the proximal ends of the left and right jaws are connected operatively to the hinge so that the hinge may facilitate pivoting motions of the jaws' distal ends between a splayed lead wire receiving position and a contracted lead wire clamping position.

Further structural components of the instant inventive adaptor comprise left and right lever arms whose proximal ends are respectively fixedly and operatively joined with the hinge and with the left and right jaws' proximal ends. Upon finger-tip actuated manipulations exerted by a physician against such lever arms to drive their upwardly extending distal ends toward each other, the hinge functions as a fulcrum with respect to the levers. Oppositely directed finger-tip pressure applied to the arms operatively levers the jaws about their common hinge fulcrum, causing them to splay to allow an upward receipt of a lead wire.

Inner or medial aspects of the left and right lever arms preferably form a wedge member receiving channel. Upon a sliding insertion of a bar configured wedge member into such channel, the left and right lever arms may be forcefully splayed away from each other, forcefully pivoting the left and right jaws about their hinge fulcrum, and forcefully inwardly or medially driving the distal ends of the jaws toward each other. Accordingly, an insertion of the wedge member into the channel formed at the vertex of the lever arms may effectively clamp a wire lead between the adaptor's jaws.

The inner faces of the distal ends of the left and right jaws preferably respectively present leftwardly opening and rightwardly opening channels which may securely nestingly receive the curved left and right faces of a lead wire, such wire extending between and longitudinally along the distal ends of the left and right jaws. Accordingly, the preferred embodiment of the instant inventive adaptor includes a quadruple of longitudinally extending channels, such channel quadruple including an upper left channel which opens rightwardly from the inner face of the left lever arm, an upper right channel which opens leftwardly from the inner face of the right lever arm, a wire receiving lower left channel which opens rightwardly at the distal end of the left jaw, and a wire receiving lower right channel which opens leftwardly at the right jaw's distal end.

The instant inventive adaptor preferably further comprises a plurality of legs which are fixedly attached to or are formed wholly with the left and right jaws. Such legs extend downwardly from the jaws' lower ends, and such legs preferably have points at their extreme lower ends. Upon placement of the points of the adaptor's legs against, for example, an upper surface of a fascia tissue layer which overlies musculature overlying a patient's spine, the physician may manually press the adaptor downwardly. Such pressure causes the legs to simultaneously pierce and extend downwardly through the fascia. Upon such pressure induced fascia piercing action, lower portions of the legs advantageously extend downwardly from the fascia's lower surface and into underlying muscle tissue. In operation of the instant inventive adaptor, both the muscle fibers underlying the fascia and the undersurface of the muscle's fascia covering constitute and are utilized as adaptor anchoring structures.

The downward extensions of the adaptor's legs into the spinal musculature are preferably retained and preserved by a plurality of extraction resisting protuberances which are fixedly attached to or formed wholly with the legs. Such protuberances may engage the under surface of the fascia and/or muscle fibers directly underlying the fascia to resist upward movement of the adaptor away from the fascia. Variously configured hooks, flanges, and arms which may extend from the legs' slide walls for enhancing frictional contact between the legs and surrounding tissues are considered to fall within the scope of the invention.

Upon piercing engagements of the legs with the fascia and/or with the underlying muscle tissue, the longitudinal end of the adaptor may be advantageously held in close proximity with a Tuohy needle lanced aperture within the facia, such aperture preliminarily receiving a longitudinal and acutely angled downward extension of the electrical wire lead. To allow the adaptor to perform further anchoring within such Tuohy needle lanced aperture, the longitudinal end of the adaptor preferably presents a forwardly and downwardly protruding nose component. Such nose component is preferably laterally segmented for further jaw grasping of the longitudinal extension of the wire lead, each such nose segment extending longitudinally and angularly downward toward the longitudinally adjacent Tuohy needle lanced aperture within the fascia. Downwardly directed traction or pulling forces exerted by the legs and their extraction resisting protuberances upon the overlying jaws advantageously pull the adaptor downwardly against the fascia, such downward traction flexibly downwardly deflecting or cupping the fascia at the implantation site. Such downward deflection of the fascia advantageously causes lower aspects of the adaptor's nose to depress downwardly into the Tuohy needle lanced aperture, resulting in further anchoring of the adaptor at and within such aperture.

The leg traction induced downward deflection of the fascia at the adaptor's implantation site additionally tends to downwardly skew the oppositely longitudinal edge of the Tuohy needle aperture downwardly, such downward skewing relatively upwardly skewing the aperture's longitudinal edge. Such leg traction induced aperture skewing advantageously allows the longitudinal edge of the aperture for to function as a slide stop. Leg traction actuated depression of the adaptor's nose into the Tuohy needle aperture positions the upper/forward aspect of the nose immediately oppositely longitudinally from the upwardly skewed longitudinal end of the Tuhoy needle aperture, allowing the nose to stop against the aperture's edge. Accordingly, the instant inventive adaptor's nose component, in combination with the traction function of the adaptor's legs, performs dual anchoring functions by means of the nose's downward extension into the Tuohy needle aperture in the fascia, and by means of the nose's contact in the manner of a slide stop with the aperture's longitudinal edge.

Accordingly, objects of the instant invention include the provision of an anchoring adaptor which incorporates structures as described above and which arranges those structures in relation to each other as described above for the performance of the above-described functions. Other and further functions and advantages of the invention will become known to those skilled in the art upon review of the appended drawings and upon reading the detailed description which follows.

### BRIEF DESCRIPTION OF DRAWINGS

FIG. 1 is a perspective view of an adaptor for percutaneous anchoring of a wire lead component of an electrical nerve stimulation assembly.

FIG. 2 re-depicts the structure of FIG. 1, the view of FIG. 2 showing a lock bar component of FIG. 1 in its assembled location, the view further showing a wire lead component of the electrical nerve stimulation assembly received within and grasped by the adaptor.

FIG. 3 is a sectional view as indicated in FIG. 1.

FIG. 4 is an alternative sectional view, as indicated in FIG. 1.

FIG. 5 re-depicts the structure of FIG. 4, the view of FIG. 5 showing an installed lock bar component.

FIG. 6 re-depicts the structure of FIG. 4, the view of FIG. 6 further showing the wire lead of FIG. 2, and showing the adaptor at a surgically implanted location within a patient's back.

FIG. 7 re-depicts the structure of FIG. 6, the view of FIG. 7 further showing the lock bar at its installed position.

FIG. 8 presents an alternate configuration of the structure of FIG. 3.

FIG. 9 is a magnified view of a portion of the structure of FIGS. 6 and 7, as show in FIGS. 6 and 7.

### DETAILED DESCRIPTION OF THE INVENTION

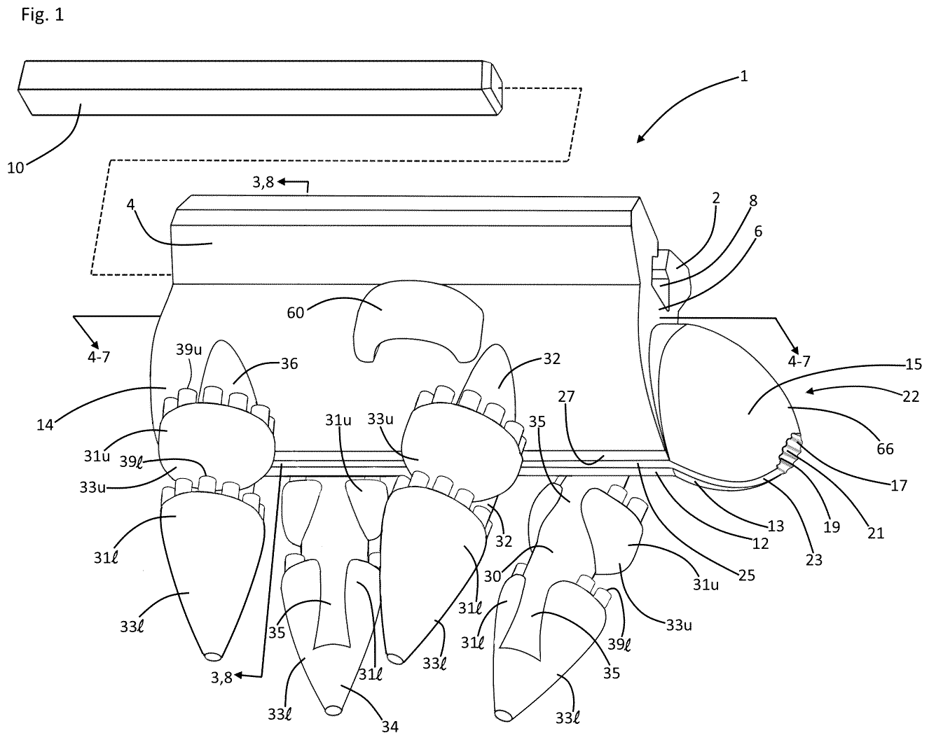

Referring to drawing FIG. 1, a preferred embodiment of the instant inventive adaptor for percutaneous anchoring of a lead wire component of an electrical nerve stimulation assembly is referred to generally by reference arrow 1. The adaptor 1 comprises left and right jaws 12 and 14. According to the view of FIG. 1, such jaws 12 and 14 have a longitudinal extension with their longitudinal or forward ends positioned rightwardly according to the view, and with their oppositely longitudinal or rearward ends positioned leftwardly according to the view. Lower, ventral, or anterior ends of the jaws 12 and 14 are positioned downwardly according to the view of FIG. 1, and their dorsal, upper, or posterior ends are oriented upwardly in the view. Consistently with an anatomical jaw, the distal ends of the jaws 12 and 14 reside at their lower or ventral ends, their pivoting proximal ends being relatively positioned at their upwardly opposite or dorsal ends.

The proximal ends of the left and right jaws 12 and 14 are preferably hingedly or pivotally interconnected by a plastic living hinge 6, such hinge being representative of other types of hinges such as pin-eye-sleeve hinges (not depicted within views) or hook and slot hinges (not depicted within views). While various commonly known, types and classes of hinges may be incorporated, and are considered to fall within the scope of the invention, the depicted living hinge 6 is preferred because of such hinge has a plastic memory characteristic which allows it to act as a biasing spring. The living hinge 6 advantageously normally moves the jaws' distal ends toward each other from a laterally splayed lead wire receiving position to a contracted lead wire holding position.

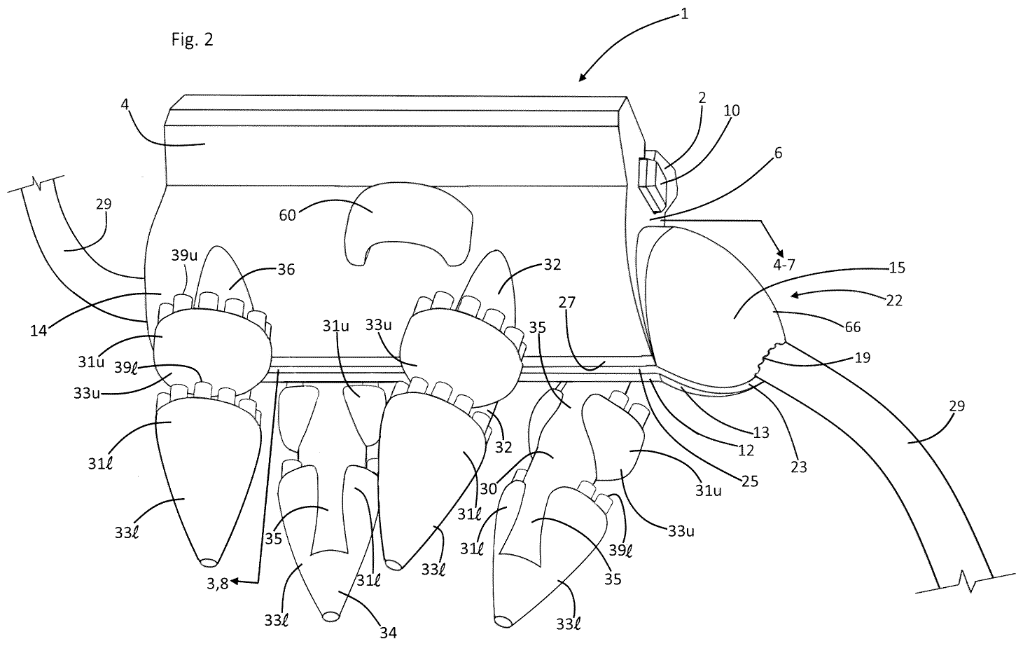

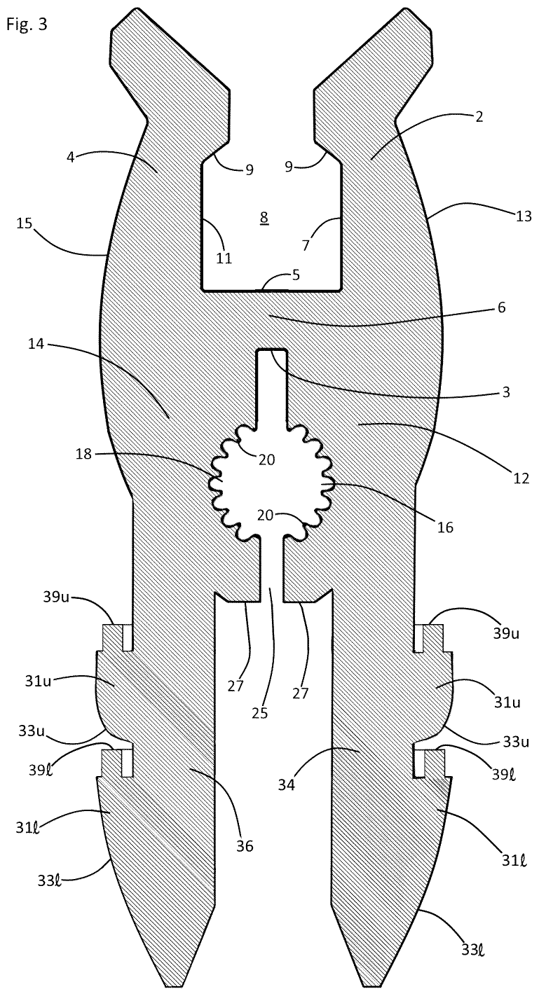

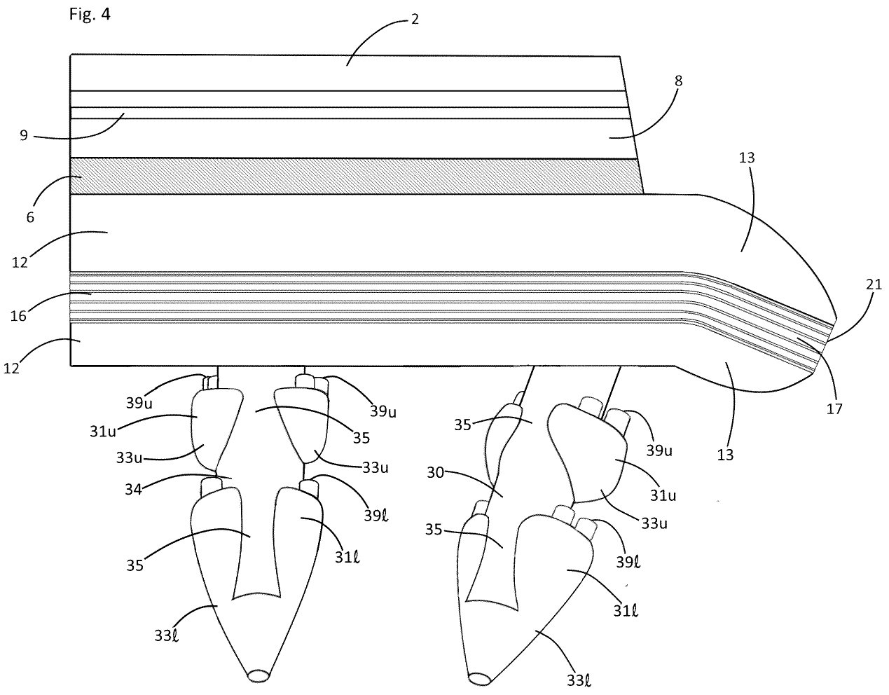

Referring to simultaneously to FIGS. 1-4, the rightward face of the left jaw 12 preferably presents a longitudinally extending and rightwardly opening lead wire receiving channel 16. As shown in FIG. 3, the leftward face of the right jaw 14 mirroringly includes a longitudinally extending and leftwardly opening lead wire receiving channel 18. Such lead wire receiving channels 16 and 18 preferably present series of longitudinally extending ridges 20 which may securely impinge against and may securely frictionally grasp the sides of an electric lead wire (e.g. FIG. 2's lead wire 29) which may be nestingly received within the channels 16 and 18.

The instant inventive adaptor 1 preferably further comprises left and right lever arms 2 and 4 which are respectively fixedly attached to or formed wholly with the proximal ends of the adaptor's left and right jaws 12 and 14. In a preferred embodiment, the left and right lever arms 2 and 4 extend longitudinally and co-extensively with the left and right jaws 12 and 14. The adaptor's preferably provided left and right lever arms 2 and 4, may be initially positioned at normal positions shown in FIGS. 1 and 3. While the jaws 12 and 14 reside at their normal positions, the living hinge 6 may hold their rightwardly and leftwardly opening channels 16 and 18 at a contracted position which allows their longitudinally extending ridges 20 to lightly impinge inwardly against the lead wire 29. However, while the jaws 12 and 14 occupy their normally contracted positions, the lower gap 25 between their lower distal ends may not be not wide enough to permit an upward insertion of such lead wire therethrough.

To facilitate an upward receipt of the lead wire 29 through the gap 25 and into the channels 16 and 18, a physician operator of the instant inventive adaptor 1 may tacitly grasp the extreme distal or upper ends of the arms 2 and 4 between the tips of his or her index finger and thumb, thereby compressing and driving the arms' ends toward each other. Application of such finger-tip pressure against the lever arms' ends provides ergonomic advantages in addition to the physician's use of the arms 2 and 4 as a handle for precise maneuvering and positioning of the adaptor. Precise positioning of the adaptor with respect to the lead wire is needed while the adaptor is splayed, and the arms 2 and 4 simultaneously facilitate both the handle function and the jaw splaying function.

Out-turned ridges which are formed at the extreme upper left and upper right distal ends of the lever arms 2 and 4 frictionally engage the physician's finger-tips, allowing them to splay the jaws 12 and 14 while assuring secure holding of the adaptor during guidance of the wire 29 between the jaws.

Following insertion of the lead wire 29 upwardly through the widened gap 25, the operator may release the finger-tip pressure from the distal ends of lever arms 2 and 4, allowing the plastic memory of the living hinge 6 to return the jaws 12 and 14 to their normal lead wire compressing and holding positions.

As shown in FIG. 3 it may be seen that the living hinge 6 has a lower wall 3 and an upper wall 5, that the left lever arm 2 has a left wall 13 and a right wall 7, and that the right lever arm 4 has a right wall 15 and a left wall 11. While such walls 5, 7, and 11 function as structural wall components of the living hinge 6, of the lever arm 2, and of the lever arm 4, such walls additionally function in combination with each other to form a lock bar receiving channel 8. Referring further to FIGS. 1 and 2, where the adaptor includes such channel 8, the adaptor preferably further comprises a lock bar 10 which has a lateral cross sectional profile closely matching the lateral cross-sectional profile of such channel. Where, for example, the diameter of the circular channel which is formed by the leftwardly and rightwardly opening channels 16 and 18 is 1.3 millimeters (matching a common diameter of lead wire 29), the lateral displacement of the lock bar channel's walls 7 and 11 may suitably be approximately 2.6 millimeters. Upon provision of such exemplary diameter and dimensions of the channels 8, 16, 16 and 18, the lateral dimension of the lock bar 10 is preferably slightly greater than the width of channel 8 so that, upon a longitudinal extension and sliding receipt of the lock bar 10 into and within channel 8, the left and right walls of the lock bar 10 may compressively drive the lever arms 2 and 4 away from each other, thereby laterally splaying such arms to forcefully pivot the left and right jaws 12 and 14 toward each other. In such example, the lateral dimension of the lock bar 10 may be 2.8 mm.

Provided that the lead wire 29 has been preliminarily received within and extended along channels 16 and 18, such sliding insertion of the lock bar 10 into the channel 8 advantageously causes the left and right jaws 12 and 14 to contract and pivot toward each other about the living hinge 6. Such pivoting and contracting jaw motions advantageously cause the radially inner ends of the channels' ridges 20 to impinge against and securely hold the lead wire between the jaws.

The longitudinal end of the lock bar 10 preferably has chamfered or beveled edges which allow the bar's longitudinal end to be easily manually pressed into the oppositely longitudinal opening of the channel 8. To secure the lock bar 10 against any unintended upward displacement or extraction from the channel 8, retainer ridges 9 are provided, such ridges overlying upper surfaces of the lock bar 10 upon the bar's insertion into channel 8.

In a preferred embodiment, a plurality of legs 30, 32, 34, and 36 is fixedly attached to or formed wholly with the left and right jaws 12 and 14, such legs having tissue piercing pointed lower ends. The legs 30, 32, 34, and 36 have extraction resisting protuberances 31u and 311 which, as shown in FIG. 3, have arcuately curved lower walls 33u and 331, and have substantially flat and upwardly facing upper walls or shelves 39u and 39l. Upon a physician's application of finger-tip pressure against the upper edges of the lever arms 2 and 4, the lower pointed ends of the legs 30, 32, 34, and 36 may initially contact underlying tissue, such as, for example, an upper surface of a fascia covering of musculature which overlies the spine of a patient's back. Upon the physician's further exertion of such downward pressure, the legs' pointed ends may pierce such facia layer, causing the pointed ends to traverse the fascia and to enter muscle tissue immediately underlying the fascia. The curved lower surfaces 33u and 331 of the protuberances 31u and 311 successively act as mandrels which enlarge such fascia apertures, such curved lower surfaces 33u and 33l easing and allowing the legs' downward passages. Upon complete downward insertions of the legs 30, 32, 34, 36 through such fascia layer, the flat shelf edges 39l of the lowermost protuberances 311 may advantageously engage and hook against surrounding muscle fibers which underlie the fascia.

As can be seen in FIG. 3, the shelves 39u of the uppermost protuberances reside at an elevation below that of the extreme lower surfaces or edges 27 of the left and right jaws 12 and 14. In a preferred embodiment, such differences in elevation are between 1 millimeter and 1.25 millimeter, such elevation differentials allowing the upper shelf surfaces 39u to hook against the fascia's undersurface while the jaws' lower edges 27 bear against the fascia's upper surface. Such vertical dimensional displacement of the upper shelves 39u below the jaws' lower edges 27 may securely “sandwich” or interstitially hold a fascia layer which is typically appropriately 1 millimeter thick.

Accordingly, the uppermost protuberances 31u advantageously engage the fascia tissue while the lowermost protuberances 311 engage underlying muscle fibers. Such dual modes of tissue engagement provide firm and secure anchoring of the adaptor 1 at a selected implantation site.

As shown in FIGS. 1-3, the legs' protuberances 31u and 311 are preferably specially configured to incompletely circumferentially extend in “C” configurations about the legs 30, 32, 34, and 36, each of the protuberances 31u and 311 having a void inner or medial section 35 with omits any radially outward extension from the leg. By providing such protuberance free medial voids 35, the protuberances 31u and 311 advantageously tend to medially bias or inwardly contract the jaws 12 and 14 toward each other during their simultaneous implantations, thereby enhancing the jaws' wire lead clamping and grasping function. In absence of such inner voids 35, the legs 30, 32, 34, and 36 may undesirably splay the jaws 12 and 14 upon manual downward driving insertions of the legs through the fascia.

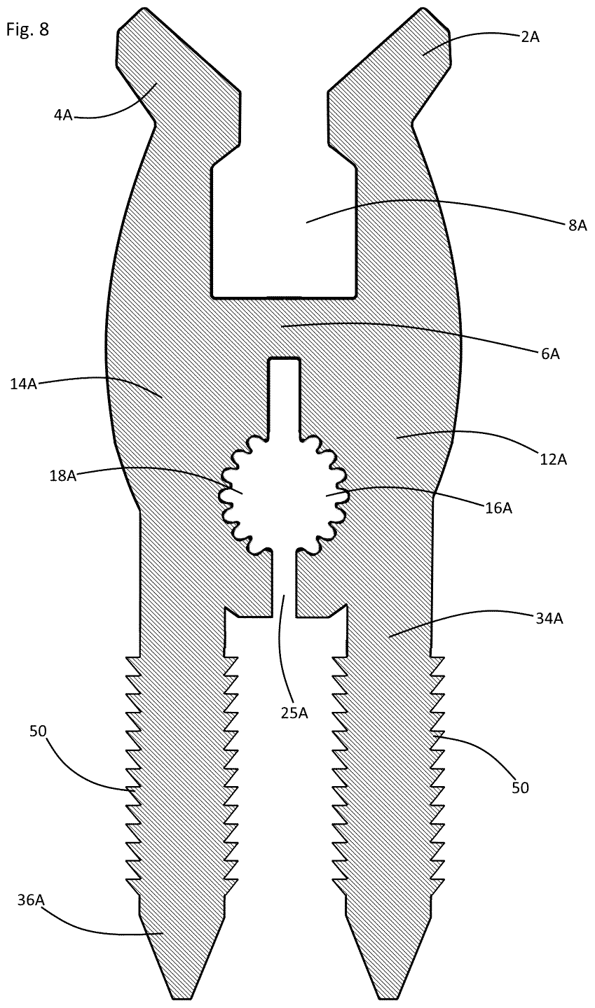

Referring simultaneously to FIGS. 3 and 8, each structure appearing in FIG. 8 which is identified by a reference numeral having a suffix “A” is configured similarly with similarly numbered structures appearing in FIG. 3. In the FIG. 8 alternative configuration, the leg anchoring protuberances are configured as annularly extending “V” ridges 50. In a further alternative embodiment, portions the side walls of the legs 30, 32, 34, 36, of the FIGS. 1-3 embodiment which are not occupied by one of the shelf protuberances, 31u or 311, may include annularly extending “V” ridges similar to ridges 50. Accordingly, for enhanced frictional contact and anchoring of the legs 30, 32, 34, and 36 within surrounding tissues, the legs may present both protuberances of the type represented by protuberances 31u and 311 and protuberances of the type represented by protuberances 50. The shelf presenting extraction resisting protuberances 31u and 311 of the FIGS. 1-3 embodiment and the annular “V” ridge protuberances 50 of the FIG. 8 embodiment are considered as being representative of numerous other extraction resisting members which may be operatively attached to and may extend from the side walls of the adaptor's legs, such as angled tabs, arms, barbs, and the like (not depicted within views).

Referring to FIGS. 1-5, the instant inventive adaptor 1 preferably further comprises a nose component which is referred to generally by reference arrow 22. In such preferred embodiment, the adaptor's nose 22 comprises left and right halves 13 and 15, the nose's left half 13 constituting a longitudinal extension of the longitudinal end of the adaptor's left jaw 12, and the nose's right half 15 mirroringly constituting a longitudinal extension of the longitudinal end of the adaptor's right jaw 14. Similarly with the presentations at the inner surfaces of the left and right jaws 12 and 14 of left and right longitudinally extending channels 16 and 18, the left and right nose segments 13 and 15 present mirroring left and right channels 17 and 19, such channels respectively forming left and right longitudinal and downward extensions of the jaws' left and right channels 16 and 18.

The longitudinal openings of the nose's left and right channels 17 and 19 form a substantially circular outlet port 21 from which the lead wire 29 may extend longitudinally and angularly downwardly. In the preferred embodiment, such channels 17 and 19 along with the nose's left and right halves 13 and 15 are canted or angled downwardly at an angle “a” with respect to the flat lower surfaces 27 of jaws 12 and 14, and in such embodiment, the forward extension of the nose 22 in combination with its downwardly deflected angle “a” may downwardly (or dorsally/ventrally) position the outlet port 21. Such downward port deflection advantageously allows the upper end of such port to reside at an elevation at or below the elevation of the lower ends of the jaws' channels 16 and 18, and allows the lower end of such port 21 to reside at an elevation at or below that of the jaws' lower surfaces 27. The lower aspects of the nose's halves 13 and 15 are preferably downwardly arcuately curved while the upper aspects of such halves are preferably both upwardly and longitudinally arcuately curved, the functions of such curved nose surfaces being described below.

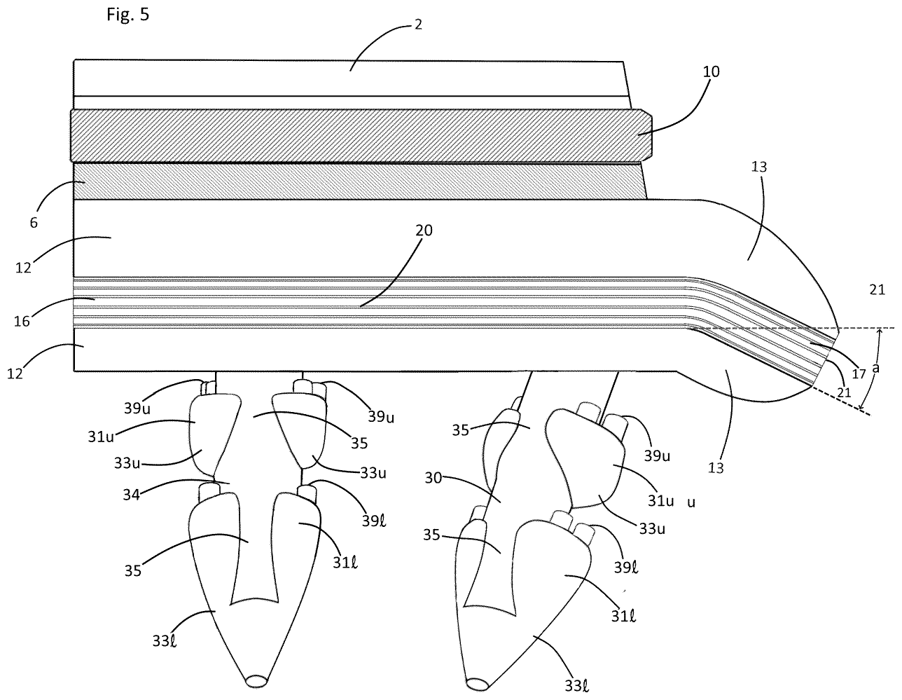

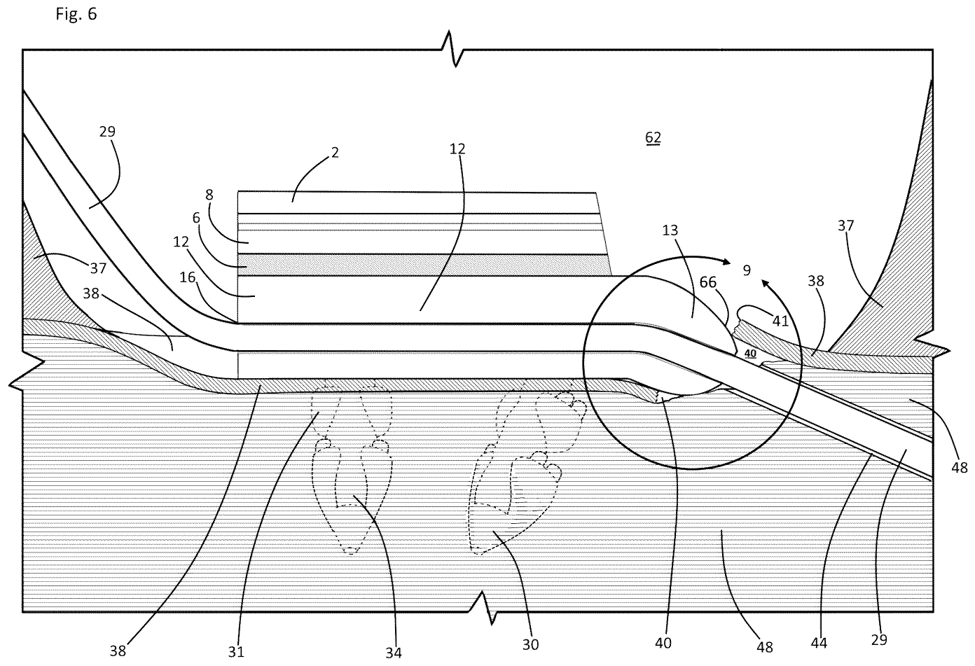

In use of the instant inventive adaptor 1, an insulated bundle of electrically conductive wires 29 may, referring to FIGS. 5 and 6, be preliminarily implanted within and extended through musculature 48 overlying the dorsal aspect of a patient's spine (not shown within view). While implantation of the adaptor at and over a patient's spine constitutes a common and typical mode of use of the adapter, such implantation site is not exclusive. The instant inventive adaptor may be utilized for anchoring a wire lead which extends along another nerve such as a lateral spinal nerve, such usage entailing implantation within and upon other tissues adjacent such spinal nerve. In the exemplary adaptor implantation which is situated at and over the prone patient's spine and spinal cord, the wire lead 29 suitably extends downwardly and forwardly at an approximate 20-degree angle within a channel 44 which is typically formed by means of a lancing insertion of a Tuohy needle or canula (not depicted within views). Such approximate 20-degree extensions of the wire lead 29 and channel 44 are preferably matched by the nose's angle “a” shown in FIG. 5, such angular orientation assisting in the adaptor's wire extending and anchoring functions.

A further longitudinal and downward extension of the wire lead 29 directs the wire between an adjacent pair of the patient's vertebrae to enter and extend along the patient's spinal canal. An electrode head (not depicted within views) which is electrically connected with individual conductors within the wire lead 29, and which is mounted at the extreme longitudinal end of such lead, may be precisely positioned by the physician at a targeted location along and over the patient's spinal cord by means of manipulations of the oppositely longitudinal extension of the wire lead 29.

Following extension and placement of such electrode head at the targeted location within the patient's spinal canal, the physician may utilize a scalpel to open a small adaptor receiving incision 62 at and over the oppositely longitudinal extension of the lead wire 29, such incision 62 traversing epidural and subdural skin layers 37. The incision 62 suitably establishes the upper surface of the patient's muscle covering facia 38 as an incision floor.

Thereafter, the physician may grasp the adaptor 1 between the tips of his or her index finger and thumb. Thereafter, the physician may press lever arms 2 and 4 toward each other, causing jaws 12 and 14 to splay away from each other, such finger-tip pressure widening the adaptor's gap 25 for receipt of the wire lead 29. Thereafter, the physician may move the wire lead 29 into the widened gap 25, causing the wire lead 29 to extend longitudinally along and to nest within channels 16, 17, 18, and 19. Thereafter, the physician may release the finger-tip pressure from the lever arms 2 and 4, allowing plastic memory of the living hinge 6 to close or contract the jaws 12 and 14. Upon such hinge actuated closure, the jaws may advantageously loosely hold the left and right aspects of the lead wire 29 within the channels 16, 17, 18, and 19.

Thereafter, the physician may position the adaptor's nose 32 so that it upwardly overlies the Tuohy needle pierced aperture 40 within the fascia 38. Thereafter, the physician may manually press and drive the adaptor's pointed legs 30, 32, 34, and 36 downwardly against the fascia 38, causing the legs to pierce therethrough. Such leg piercing actions allow the legs' protuberances 311 and 31u to successively engage the fascia 38 and the underlying muscle fibers 48 in the manner of hooks or stop flanges.

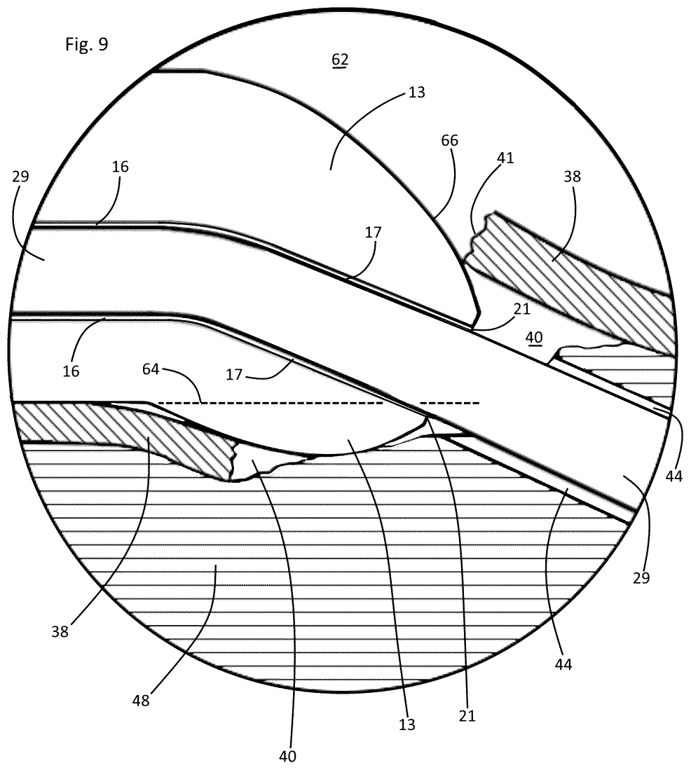

Substantially simultaneously with such leg driving and fascia piercing action, the curved lower surface of the nose 22 is caused to extend downwardly into the fascia aperture 40, allowing the nose's curved upper and longitudinal surfaces 66 to function in the manner of a slide stop against the aperture's relatively upwardly deflected longitudinal edge 41. As shown in the magnified view of FIG. 9, the approximate 20 degree downward angles of the adaptor's nose 22 and of the nose's channels 17 and 19 advantageously position the lower end of the longitudinal wire port 21 below the lower ends 27 of jaws 12 and 14, such positioning of port 21 downwardly deflecting such port into aperture 40. Such downward positioning of the port 21 also advantageously allows the nose's curved upper and forward surface 66 to function in the manner of a slide stop against the the longitudinal edge 41 of aperture 40. Such slide stop's abutting components 66 and 41 may advantageously resist slippage of the adaptor 1 and the wire lead 24 in the longitudinal direction.

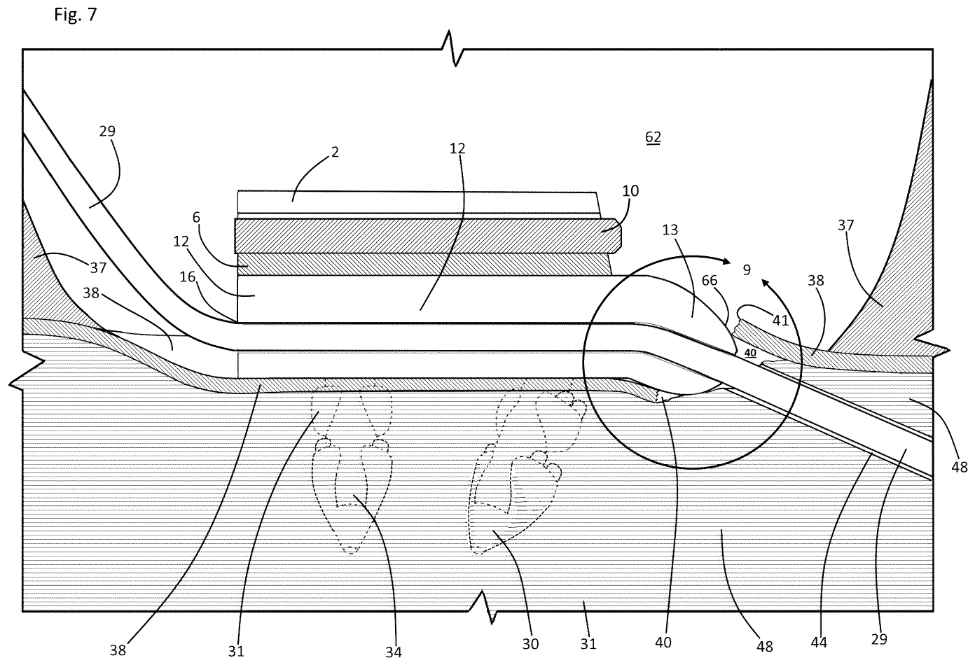

FIGS. 6 and 7 show that, at the site of implantation of the adaptor, the fascia 38 dips or deflects downwardly below the fascia's surrounding surfaces, effectively cupping the fascia at the adaptor implantation site. Such downward fascia deflection is effected by means of the downward traction which is exerted by the legs 30, 32, 34, and 36 against the undersurfaces 27 of the adaptor. Such leg traction draws both the adaptor and the immediately underlying fascia downwardly below the elevation of the surrounding fascia tissues. Concurrently with such downward fascia deflection, the Tuohy needle pierced fascia aperture 40 is distorted or deflected, as depicted, so that its longitudinal edge is relatively high with respect to its downwardly deflected oppositely longitudinal edge. Such aperture distortion effectively positions the aperture's longitudinal edge 41 for performance of its function, as discussed above, as a slide stop, Accordingly, the adaptor's legs 30, 32, 34, and 36 advantageously perform multiple anchoring functions, via anchoring within their own leg pierced facia apertures, via downward anchoring the adaptor's nose 22 within the Tuohy needle pierced aperture 40, and via positioning of such nose for slide stopping contact with the longitudinal edge of such aperture.

While the adaptor 1 is initially implanted and assembled within incision 62 as depicted in FIG. 6, the longitudinal position of the wire lead 29 may be adjusted via small longitudinal or oppositely longitudinal sliding motions along the channels 16, 18, 17 and 19. Following such wire position adjustments, and upon establishment of a final precisely selected position of the lead wire 29 within the anchored adaptor 1, the physician may slidably insert the lock bar 10 within lock bar channel 8 as depicted in FIG. 7. The wedge functioning presence of the lock bar 10 within the channel 8 forcefully splays lever arms 2 and 4 away from each other, simultaneously forcefully contracting jaws 12 and 14 toward each other in pivoting motions about living hinge 6. Such splaying and contracting actions of the arms and jaws 2, 4, 12, and 14 securely clamp the lead wire 29 between the jaws 12 and 14 and within the jaws' channels 16, 17, 18, and 19.

Following the insertion of the lock 10 into channel 8, the lead wire 29 is securely held and positioned at the selected position depicted in FIG. 7 by the engagements of the legs 30, 32, 34, and 36 with fascia 28 and muscle fibers 48, and by the engagements of the nose 22 with the Tuohy needle pierced aperture 40. The anchoring effect of the angular downward extension of the adaptor's nose 22 into fascia aperture 40 further secures the adaptor and its clamped lead wire by resisting longitudinal slippage of the adaptor while the legs resist both longitudinal and vertical slippage.

Following implantation of the adaptor as depicted in FIG. 7, the physician may tactilely assess the strength of the implantation. In many circumstances, the leg and nose anchored adaptor will be found to be sufficiently secure. Notwithstanding, for additional anchoring security against lead wire slippage the physician may utilize suitably provided left and right loops 60 as suture attachment points. Sutures may be extended by the physician through such loops 60 and through surrounding tissues for further secure anchoring of the adaptor 1.

Thereafter the incision 62 may be surgically closed. In a typical implantation procedure, the oppositely longitudinal extension of the lead wire 29 communicates with a subcutaneously implanted electrical pulse generating unit (not depicted within views).

While the principles of the invention have been made clear in the above illustrative embodiments, those skilled in the art may make modifications to the structure, arrangement, portions, and components, of the invention without departing from those principles. Accordingly, it is intended that the description and drawings be interpreted as illustrative and not in the limiting sense, and that the invention be given a scope commensurate with the appended claims.

1. An adaptor for percutaneous anchoring of a wire lead component of an electrical nerve stimulation assembly, said adaptor comprising:
a. A left jaw having a proximal end, a distal end, and right side;
b. A right jaw having a proximal end, a distal end, and a left side;
c. A left arm having a proximal end, a distal end, and a right side;
d. A right arm having a proximal end, a distal end, and a left side;
e. An upper left channel opening rightwardly at the left arm's right side;
f. An upper right channel opening leftwardly at the right arm's left side;
g. A lower left channel opening rightwardly at the left jaw's right side;
h. A lower right channel opening leftwardly at the right jaw's left side; and
i. A hinge fixedly attached to or formed wholly with the proximal ends of the left arm, the right arm, the left jaw, and the right jaw.

2. The adaptor of claim 1, further comprising a wedge fitted for, upon insertion between the left and right arms, nesting within the upper left and upper right channels.

3. The adaptor of claim 1, wherein the arms and jaws are composed of plastic, and wherein the hinge comprises a plastic living hinge.

4. The adaptor of claim 1, further comprising a plurality of legs, wherein each leg is fixedly attached to and extends downwardly from one of the jaws.

5. The adaptor of claim 4, wherein each leg has a pointed lower end.

6. The adaptor of claim 5, further comprising a plurality of tissue engaging protuberances, wherein each tissue engaging protuberance is fixedly attached to or formed wholly with one of the legs.

7. The adaptor of claim 6, wherein each tissue engaging protuberance extends circumferentially about one of the legs.

8. The adaptor of claim 7, wherein said each tissue engaging protuberance has a medial void section.

9. The adaptor of claim 7, wherein said each tissue engaging protuberance has a shelf configured upper end.

10. The adaptor of claim 7, wherein said each tissue engaging protuberance has an arcuately curved lower end.

11. An adaptor for percutaneous anchoring of a wire lead component of an electrical nerve stimulation assembly, said adaptor comprising:
a. A left jaw having a proximal end, a distal end, and a right side;
b. A right jaw having a proximal end, a distal end, and a left side;
c. A left channel opening rightwardly at the left jaw's right side;
d. A right channel opening leftwardly at the right jaw's left side; and
e. A hinge fixedly attached to or formed wholly with the left and right jaws' proximal ends.

12. The adaptor of claim 11, wherein the hinge comprises a living hinge.

13. The adaptor of claim 12, further comprising a plurality of friction ridges within the left and right channels.

14. The adaptor of claim 13, further comprising a plurality of legs, wherein each leg is fixedly attached to and extends downwardly from the distal end of one of the jaws.

15. The adaptor of claim 14, wherein each leg has a pointed lower end.

16. The adaptor of claim 15, further comprising a plurality of tissue engaging protuberances, wherein each tissue engaging protuberance extends annularly about one of the legs.

17. The adaptor of claim 16, wherein each tissue engaging protuberance comprises a shelf configured upper end.

18. The adaptor of claim 16, wherein said each tissue engaging protuberance has an arcuately curved lower end.

19. An adaptor for percutaneous anchoring of a wire lead component of an electrical nerve stimulation assembly, said adaptor comprising:
a. A left jaw having a longitudinal end, a proximal end, a distal end, and a right side;
b. A right jaw having a longitudinal end, a proximal end, a distal end, and a left side;
c. A first left channel opening rightwardly at the left jaw's right side;
d. A first right channel opening leftwardly at the right jaw's left side;
e. A left nose member having a longitudinal end, the left nose member being fixedly attached to and extending longitudinally from the longitudinal end of the left jaw;
f. A right nose member having a longitudinal end, the right nose member being fixedly attached to and extending longitudinally from the longitudinal end of the right jaw;
g. A second left channel opening at the left nose member, the second left channel communicating with the first left channel;
h. A second right channel opening at the right nose member, the second right channel communicating with the first right channel; and
i. A hinge fixedly attached to or formed wholly with the left and right jaws' proximal ends.

20. The adaptor of claim 19, wherein the left and right nose members extend downwardly at an acute angle from the longitudinal ends of the left and right jaws.

21. The adaptor of claim 20, wherein the first left and first right channels have longitudinal ends, and wherein the second left and second right channels respectively extend downwardly at acute angles from said channels' longitudinal ends.

22. The adaptor of claim 21, wherein the second left and second right channels have longitudinal ends, and further comprising a lead output port, wherein the lead output port opens at said longitudinal ends.

23. The adaptor of claim 22, wherein the left nose member has a left arcuately curved upper edge, and wherein the right nose member has a right arcuately curved upper edge.

24. The adaptor of claim 23 wherein the left and right jaws have lower ends positioned at a first elevation, wherein the lead output port has a lower end positioned at a second elevation, and wherein said second elevation is lower than said first elevation.

25. The adaptor of claim 19, further comprising a plurality of tissue anchoring legs, each tissue anchoring leg being fixedly attached to and extending downwardly from one of the jaws.

26. The adaptor of claim 25, wherein each tissue anchoring leg is adapted for downwardly biasing the left and right nose members' longitudinal ends.
